(19) United States
(12) Patent Application Publication
(10) Pub. No.: US 2017/0014138 A1
(43) Pub. Date: Jan. 19, 2017

(54) INSTRUMENT FOR POSITIONING OSTEOTOMY TOOL-HOLDER BASEPLATES

(71) Applicant: OSTESYS, Plouzane (FR)

(72) Inventors: Pierre-Yves HUET, LOCMARIA-PLOUZANE (FR); Guillaume DARDENNE, MONTREUIL LE GAST (FR); Eric STINDEL, LOCMARIA-PLOUZANE (FR)

(73) Assignee: OSTESYS, Plouzane (FR)

(21) Appl. No.: 15/124,012

(22) PCT Filed: Feb. 19, 2015
(86) PCT No.: PCT/EP2015/053509

§ 371 Date: Sep. 6, 2016

(30) Foreign Application Priority Data
Mar. 7, 2014 (FR) 1451885

(51) Int. Cl. A61B 17/15; A61B 34/20; A61B 17/80; A61B 17/88

(52) U.S. Cl. CPC A61B 17/151; A61B 17/8872; A61B 34/20; A61B 17/8095; A61B 2090/3983

(57) ABSTRACT

The invention relates to an instrument (1) for positioning osteotomy tool-holder baseplates (E1, E2).

According to the invention, the instrument contains two supports (2, 3) of said baseplates, connected together by a connecting part (4) in a semi-rigid material allowing relative displacement of one of the supports (2, 3) with respect to the other (3,2).

The invention lies within the field of osteotomy.

The present invention relates more precisely to an instrument for positioning osteotomy tool-holder baseplates.

Arthritis is a mechanical anomaly involving deteriorations of joints, comprising the articular cartilage and the subchondral bone.

This pathology can be treated through various methods, the most important being the total replacement of the pathological joint by a prosthetic implant. This method, however, is very degenerative, invasive and sometimes traumatic for the patient.

When this illness is the consequence of a misalignment of a limb, it can be slowed or treated by osteotomy.

This less invasive surgical procedure has the purpose of rebalancing the forces exerted on the pathological joint by realigning the articular centres of the limb (for example the hip, knee, ankle centres for the lower limb or the shoulder, elbow and wrist centres for the upper limb). A partial section is carried out on one of the two bones forming the limb (for example the femur or the tibia for the lower limb, or the humerus or the radius for the upper limb).

The realignment is carried out by carrying out a rotation of the two bone segments, partially cut away, around a rotation point, often called a hinge, resulting from this partial cutting. Once the alignment is obtained, these two bone segments are held in the desired position thanks to an implant in the form of a shim positioned inside the opening resulting from the distraction (spreading) of the two bone segments.

This operative technique sometimes requires positioning various tools on either side of this future cut line, such as for example: a target including markers, the positions and orientations whereof can be determined by a 3D localizer, a bone cutting guide, distracters (separators) or a milling guide.

To accomplish this, it is necessary to previously attach to these two bone segments situated on either side of the future cut line, a baseplate which serves to support these tools.

The invention thus has the goal of proposing an instrument allowing correct positioning of these baseplates, at a precise distance from one another, while adapting to the morphology of the bone at the point where they must be positioned, the bone generally not being planar.

In a particular application to the osteotomy of the tibia, the invention has particularly as an objective to propose an instrument which guarantees correct positioning of the baseplates at the anterior tibial tuberosity.

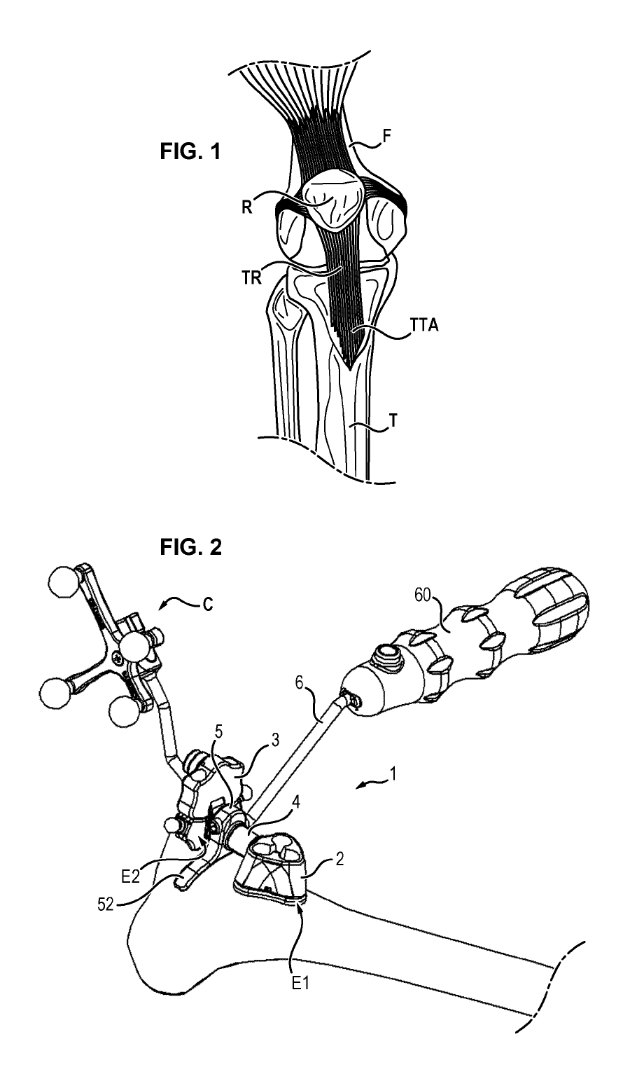

This tuberosity, referenced TTA, is visible in the appended FIG. 1.

To this end, the invention relates to an instrument for positioning osteotomy tool-holder baseplates.

In conformity with the invention, it comprises two supports of said baseplates, connected with each other through a connecting part in a semi-rigid material allowing relative displacement of one of the supports with respect to the other.

According to other advantageous and non-limiting features of the invention, taken alone or in combination:
the instrument comprises a positioning part provided with a tab for insertion under a tendon;
said positioning part is in two portions assembled by means allowing their disassembling, the insertion tab being secured to one of these two portions;
said positioning part is positioned between one of the baseplate supports and the connecting part;
the instrument comprises a handling rod.
said handling rod is attached to the positioning part.
the connecting part comprises an elastomeric body provided at both of its ends with an attachment member on each of both supports.
said attachment member is a threaded rod or a tapped bore.
the support is provided with means for attaching and temporary retaining a baseplate.
the support is provided with at least one through hole of a screw for attaching a baseplate (E1, E2) to a bone.

Other features and advantages of the invention will appear from the description which will now be given, with reference to the appended drawings which show, by way of indication and without limitation, one possible embodiment thereof.

In these drawings:

FIG. 1 is a schematic showing the knee joint,

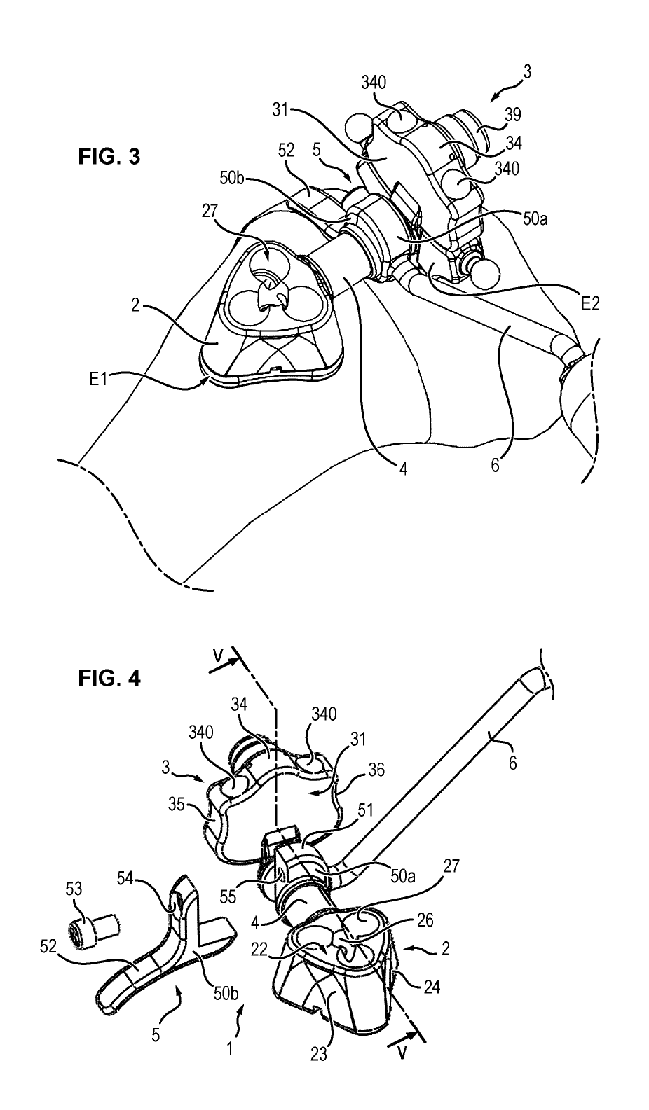

FIGS. 2 and 3 are perspective views showing the instrument conforming to the invention, from different viewing angles,

FIG. 4 is a perspective view showing the instrument conforming to the invention, with its positioning lug in the disassembled state,

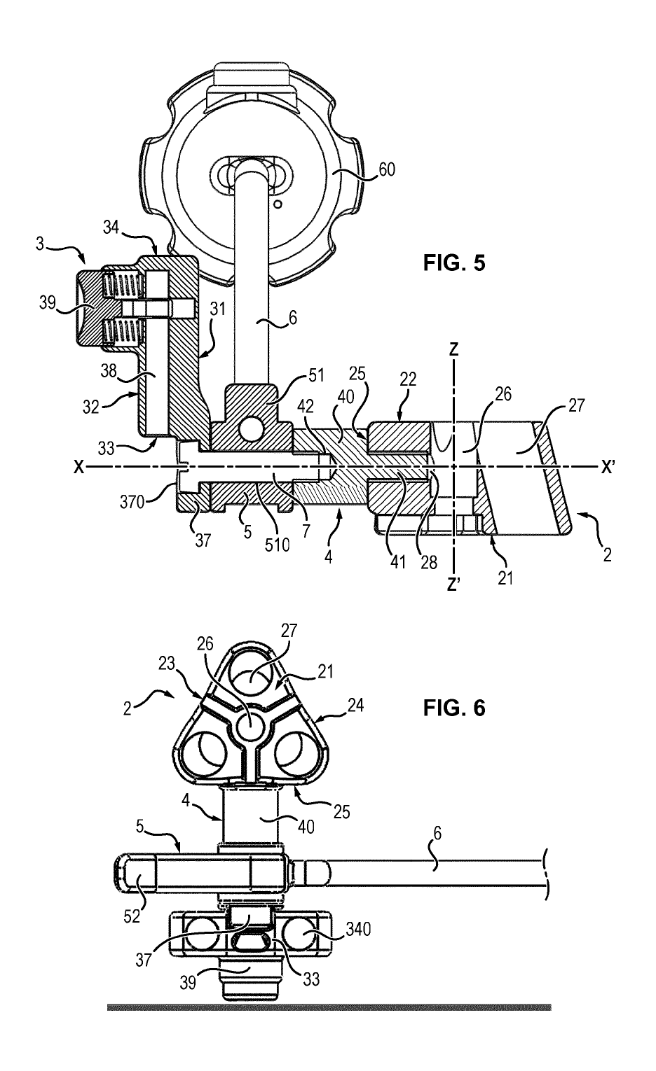

FIG. 5 is a longitudinal section view of the instrument along the section plane shown by the line V-V in FIG. 4, and

FIG. 6 is a view from below of the instrument conforming to the invention, the handle not being shown in FIGS. 3, 4 and 6.

In the remainder of the description, the instrument is described in connection with use for a tibial osteotomy. In FIG. 1, it is possible to see a tibia T, a femur F, a patella R and a patellar tendon TR. Equally, this instrument could be used for osteotomies carried out on other portions of the lower or upper limbs.

Referring to FIGS. 4 and 5 one can see that the instrument 1 for positioning osteotomy tool-holder baseplates generally comprises two supports 2 and 3 of said baseplates E1 and E2, connected to one another by a connecting part 4.

Optionally but advantageously, the instrument 1 is also provided with a positioning part 5 and/or a handling rod 6.

The two baseplate supports 2 and 3 will now be described in more detail. These are of course only examples. These supports could also have other geometric shapes, suitable for maintaining other types of baseplates, without thereby departing from the scope of the invention.

The support 2 has the general shape of a pyramidal trunk with a triangular base 21. This base 21 (or “lower face”) is planar or substantially planar. The opposite face 22 of the support 2 (called “upper face”), with a triangular shape, is also planar and parallel to the face 21.

Two sides 23 and 24 of the support 2 are inclined from the inside of the support and the face 22 toward the outside and the lower face 21, so that the upper face 22 has a smaller area than the lower face 21. Finally, the third side 25 of the pyramidal trunk is perpendicular or substantially perpendicular to the lower face 21.

The support 2 also advantageously has a bore 26 which passes through it from side to side from its lower face 21 to its upper face 22.

This bore 26 is advantageously tapped. It makes possible the introduction of a screw not shown in the figures, this screw allowing the attachment and temporary retention of a baseplate E1, as shown in FIG. 2. This baseplate E1 is a thin plate with a substantially triangular contour adopting the shape of the lower face 21.

The screw is introduced into the bore 26 so that its head is in proximity to the upper face 22 and its lower end enters a tapped bore provided for this purpose in the baseplate E1.

The operator who uses the instrument 1 can thus lift, handle and displace the baseplate E1 jointly with the instrument 1.

The support 2 also comprises at least one through hole 27, preferably three as shown in FIG. 4. These holes 27 allow the passage of a screw for attaching the baseplate E1 to a bone, for example the tibia bone T.

This hole 27 or these holes 27 are advantageously inclined with respect to the longitudinal axis Z-Z′ of the bore 26, i.e. they are inclined from inside the support 2 and the upper surface 22 toward the outside and the lower face 21.

Finally, the support 2 has a tapped bore 28 which extends along an axis X-X′ parallel to the plane of the faces 21 and 22. This bore extends from the side 25 in the direction of the bore 26 and, preferably leads into this bore 26.

The support 3 has approximately the shape of a thin rectangular parallelepiped with two large faces, one 31 oriented toward the support 2, the other opposite, referenced 32, a lower face 33, an upper face 34 and two lateral faces 35 and 36.

The support 3 has, at the intersection between these faces 31 and 33, an attachment lug 37, pierced with an opening 370 with axis X-X′ allowing passage of a screw 7.

The support 3 further has a recess 38 opening on its lower face 33. This recess 38 is intended to receive a rod or a protrusion, integral with the tool-holder baseplate E2, like that shown in FIG. 2 for example.

A pushbutton 39 cooperates with the upper end of the recess 38. The spring-loaded pushbutton 39 makes it possible to retain temporarily the baseplate E2 and, when it is actuated, to release the latter.

Finally, the support comprises at least one hole 340, preferably two for passage of a screw for attachment of the baseplate E2 to a bone.

As appears more clearly in FIG. 5, the connecting part 4 comprises a body 40, provided at both of its ends with an attachment member on each of both supports 2 and 3.

The body 40 consists of a segment, preferably solid, preferably cylindrical, of a semi-rigid material such as an elastomer. Such a material can deform elastically. By way of an example, this body 40 can be made of rubber or of silicone, preferably of the biocompatible type.

The connecting part 4 further comprises, at one of its ends, a threaded rod 41 intended to be received in the tapped bore 28 provided in the support 2.

The connecting part 4 also has, at its opposite end, a tapped bore 42 intended to receive a screw 7 so as to attach the support 3 to said connecting part 4.

The threaded rod 41 and the tapped bore 42 are preferably aligned and extend along a longitudinal axis X-X′ which also corresponds to the longitudinal axis of the body 40.

According to other variants of implementation not shown in the figures, the body 40 could be supplied, at each of its two ends, with two threaded rods 41 or conversely, with two tapped bores 42, or other attachment means making it possible to provide the connection between the part 4 and the two supports 2 and 3.

The selection of the material constituting the body 40 and the length thereof determine the degree of angular displacement that the supports 2 and 3 can occupy with respect to the longitudinal axis X-X′ of the instrument 1.

When the instrument 1 is positioned on the bone on which the osteotomy is to be performed, this angular displacement makes it possible to adapt to the morphology of the patient's bone the position of the supports 2 and 3 and therefore of the baseplates E1 and E2 which they carry. In other words, the connecting part 4 can twist so that the supports 2 and 3 can displace themselves in 3D with respect to one another. It will be noted that the position of the instrument 1 in FIG. 5 is the resting position.

The positioning part 5 comprises a body 51 of cylindrical or approximately cylindrical shape, pierced with a central bore 510 with axis X-X′. This body 51 is extended radially by a tab 52. This tab 52 is capable of being inserted between a bone and a tendon. As an example, in the case of a tibial osteotomy, this tab 52 will be inserted at the upper end of the tibia T, below the patellar tendon TR (see FIG. 1).

Advantageously, and to facilitate the removal of the instrument 1 once the baseplates E1 and E2 have been positioned on the bone, the positioning part 5 is made in two portions 50a and 50b, the latter supporting the insertion tab 52.

The two parts 50a and 50b are assembled together by means allowing their disassembling, for example using a screw 53 inserted into bores 54, 55 provided for this purpose, respectively on the parts 50b and 50a. The two parts 50a and 50b are assembled on either side of the screw 7.

Finally, advantageously, the instrument 1 can comprise a handling rod 6 attached at one of its ends to the instrument 1, preferably to the positioning part 5, for example by welding. At its opposite end, this handing rod 6 can be extended by a handle 60 (shown in FIGS. 2 and 5).

The use of the instrument 1 is the following. The two baseplates E1 and E2 are attached respectively to the supports 2 and 3 of the instrument, for example by means of screws as described previously.

The operator then positions the instrument 1 so that the baseplates E1 and E2 are disposed on either side of the future osteotomy cut line. The connecting part 4 allows the angular displacement of the support 2 with respect to the support 3. It slides the insertion tab 52 below the tendon, which participates in maintaining the position of the instrument 1.

Once the baseplates E1 and E2 are correctly positioned, they are screwed into the bone using screws which pass through the supports 2 and 3 through the holes 27 and 340.

Once the baseplates E1 and E2 are attached to the bone, the operator separates these baseplates from their respective supports 2 and 3, for example by unscrewing the screw previously introduced into the bore 26 or by activating the pushbutton 39.

If necessary, the operator separates the two portions 50a and 50b from the positioning part 5. Finally, he withdraws the instrument 1, i.e. the supports 2 and 3, the part 4, the portion 50a and the rod 6, if it is present, then subsequently, he removes the part 50b laterally.

Subsequently, the operator can position on the baseplates E1 or E2 all the tools that will be necessary for him to carry out his osteotomy, as was described previously. In FIG. 2, by way of an example, a target C can be seen positioned on the baseplate E2.

1. An instrument for positioning osteotomy tool-holder baseplates, wherein, it comprises two supports of said baseplates, connected with each other through a connecting part in a semi-rigid material allowing relative displacement of one of the supports with respect to the other.

2. The instrument according to claim 1, wherein it comprises a positioning part provided with a tab for insertion under a tendon.

3. The instrument according to claim 1, wherein said positioning part is in two portions assembled by means allowing their disassembling, the insertion tab being secured to one of these two portions.

4. The instrument according to one of the preceding claims, wherein said positioning part is positioned between one of the baseplate supports and the connecting part.

5. The instrument according to claim 1, wherein it comprises a handling rod.

6. The instrument according to claims 2 and 5, wherein said handling rod is attached to the positioning part.

7. The instrument according to claim 1 or claim 2 or claim 5, wherein the connecting part comprises an elastomeric body, provided at both of its ends with an attachment member on each of both supports.

8. The instrument according to claim 7, wherein said attachment member is a threaded rod or a tapped bore.

9. The instrument according toclaim 1 or claim 2 or claim 5, wherein the support is provided with means for attaching and temporarily retaining a baseplate.

10. The instrument according to claim 1 or claim 2 or claim 5, wherein the support is provided with at least one through hole of a screw for attaching a baseplate to a bone.
